(19) United States
(12) Patent Application Publication
(10) Pub. No.: US 2014/0107653 A1
(43) Pub. Date: Apr. 17, 2014

(54) ULTRASONIC OSTEOTOME

(71) Applicant: SUNNYTEC ELECTRONICS CO., LTD., Kaohsiung City (TW)

(72) Inventors: Chun-Pin LIN, Kaohsiung City (TW); Hao-Hung CHANG, Kaohsiung City (TW); Yi-Chao HUANG, Kaohsiung City (TW); Yu-Chih CHIANG, Kaohsiung City (TW); Hann-Kuang CHEN, Kaohsiung City (TW); Chao-Ping LEE, Kaohsiung City (TW)

(73) Assignee: SUNNYTEC ELECTRONICS CO., LTD., Kaohsiung City (TW)

(21) Appl. No.: 13/652,995

(22) Filed: Oct. 16, 2012

(51) Int. Cl. A61B 17/16

(52) U.S. Cl. 606/84

(57) ABSTRACT

An osteotome includes a connector, a first arm, a second arm, a third arm, a fourth arm and a cutting head. A coordinate system has an X axis, a Y axis, and a Z axis. The X axis, the Y axis and the Z axis are perpendicular to each other and intersect at an original point. The connector extends along the Y axis. The first arm, extending along the Y axis, connects to the second arm, extending along the plane with the X and Y axes. An angle between the first and second arms is within a range between 125 to 145 degrees. The third and fourth arms respectively extend along the Z axis and the X axis. The cutting head is parallel to the X axis and the Y axis so as to easily control the insertion angle of the osteotome for the patients with smaller face or smaller oral cavity.

### FIELD OF THE INVENTION

The present invention relates to an ultrasonic osteotome, also called as the ultrasonic bone knife or ultrasonic surgical knife, and more particularly, to an ultrasonic osteotome for patients having smaller oral cavity or smaller face, and the insertion angle of the ultrasonic osteotome is easily controlled.

### BACKGROUND OF THE INVENTION

The osteotome is used to cut dentine and enamel while the soft tissues are protected.

The conventional osteotomes are designed for western people who have larger face so that the conventional osteotomes are not suitable for the patients who have smaller face. For the patients having smaller face, the osteotome is difficult to control precisely during operation when cutting the mandible.

FIGS. 9 and 10 respectively show the first and second conventional osteotomes C1 and C2. Because the conventional osteotomes C1, C2 are designed not for the patients with smaller face or smaller oral cavity, the conventional osteotomes C1, C2 may contact the cheeks of the patients to result in uncomfortable to the patients.

When the oral cavity of the patients is small, the insertion angle for operating the conventional osteotomes C1, C2 is difficult to control. Consequently, the desired cutting area of the mandible is not precisely cut.

The conventional osteotomes C1, C2 are designed for the patients with larger face. Consequently, the use of the conventional osteotomes C1, C2 are inconvenient for the patients having smaller face or smaller oral cavity, including removal of wisdom teeth or bone transplant.

The present invention intends to provide an osteotome that improves the shortcomings of the conventional osteotomes.

### SUMMARY OF THE INVENTION

The present invention relates to an osteotome and comprises a connector, a first arm, a second arm, a third arm, a fourth arm and a cutting head. A coordinate system has an X axis, a Y axis and a Z axis. The X axis, the Y axis and the Z axis are perpendicular to each other and intersect at an original point. The connector extends along the Y axis. The first arm, extending along the Y axis, connects to the second arm, extending along the plane with the X and Y axes. An angle between the first and second arms is within a range between 125 and 145 degrees. The third and fourth arms respectively extend along the Z axis and the X axis, and the third arm connects with the second and fourth arms. The cutting head connects to the fourth arm and is parallel to the X axis and the Y axis so as to easily control the insertion angle of the osteotome for the patients with smaller face or smaller oral cavity.

In a preferred embodiment, the second arm extends toward the positive direction of the X axis to connect with the first arm, and the fourth arm extends along the negative direction of the X axis to connect with the cutting head.

In another preferred embodiment, the second arm extends toward the negative direction of the X axis to connect with the first arm, and the fourth arm extends along the positive direction of the X axis to connect with the cutting head.

Preferably, the length of the osteotome without the connector in the Y axis is 20.016 mm to 30.24 mm. The length of the osteotome in the X axis is 8 mm to 12 mm.

The length of the osteotome 1 in the Z axis is 6.4 mm to 9.6 mm. The length of the cutting head 16 in the X axis is 3.2 mm to 4.8 mm.

Preferably, the length of the osteotome without the connector in the Y axis is 25.02 mm. The length of the osteotome in the X axis is 8 mm. The length of the osteotome in the Z axis is 10 mm. The length of the cutting head in the X axis is 4 mm.

Preferably, the length of the first arm in the Y axis is 9.91 mm, and the length of the second arm in the Y axis is 10.03 mm.

Preferably, the angle between the first and second arms is 135 degrees.

The primary object of the present invention is to provide an osteotome which can reach the desired cutting area in the oral cavity of the patient with a smaller face.

The present invention is designed for the patient with a smaller face or a smaller oral cavity. The osteotome does not hit the patient's cheeks during operation.

The present invention is designed for operation to the cutting area of mandible, and the insertion angle into the patient's oral cavity is conveniently controlled.

The present invention will become more obvious from the following description when taken in connection with the accompanying drawings which show, for purposes of illustration only, a preferred embodiment in accordance with the present invention.

### BRIEF DESCRIPTION OF THE DRAWINGS

FIG. 1 is a perspective view to show a preferred embodiment of the osteotome of the present invention;

FIG. 2 is a front view to show the osteotome of the present invention;

FIG. 3 is a right side view to show the osteotome of the present invention;

FIG. 4 shows that the osteotome of the present invention is used to cut the cutting area of the mandible;

FIG. 5 shows that the osteotome of the present invention is used to cut horizontally the cutting area of the left side of the mandible;

FIG. 6 is a perspective view to show the second embodiment of the osteotome of the present invention;

FIG. 7 is a front view to show another preferred embodiment of the osteotome of the present invention;

FIG. 8 shows that the osteotome of the present invention is used to cut horizontally the cutting area of the right side of the mandible;

FIG. 9 is a perspective view to show the conventional osteotome; and

FIG. 10 is a perspective view to show another conventional osteotome.

### DETAILED DESCRIPTION OF THE PREFERRED EMBODIMENT

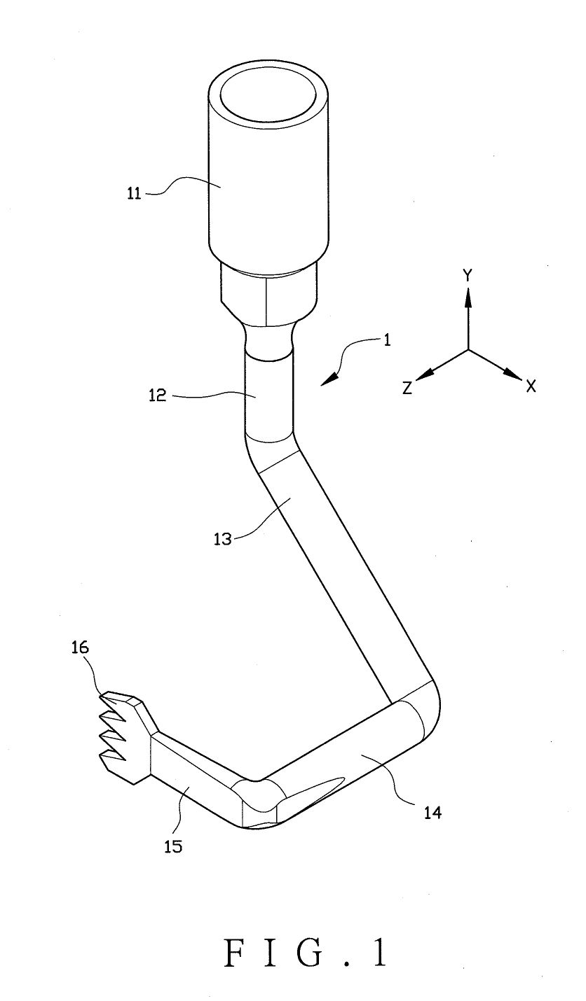

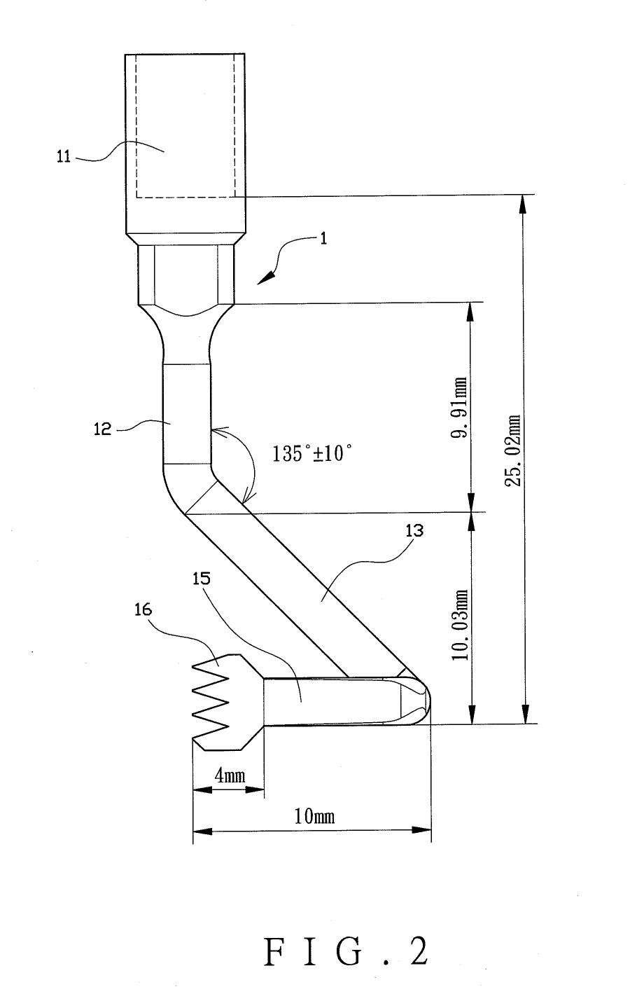

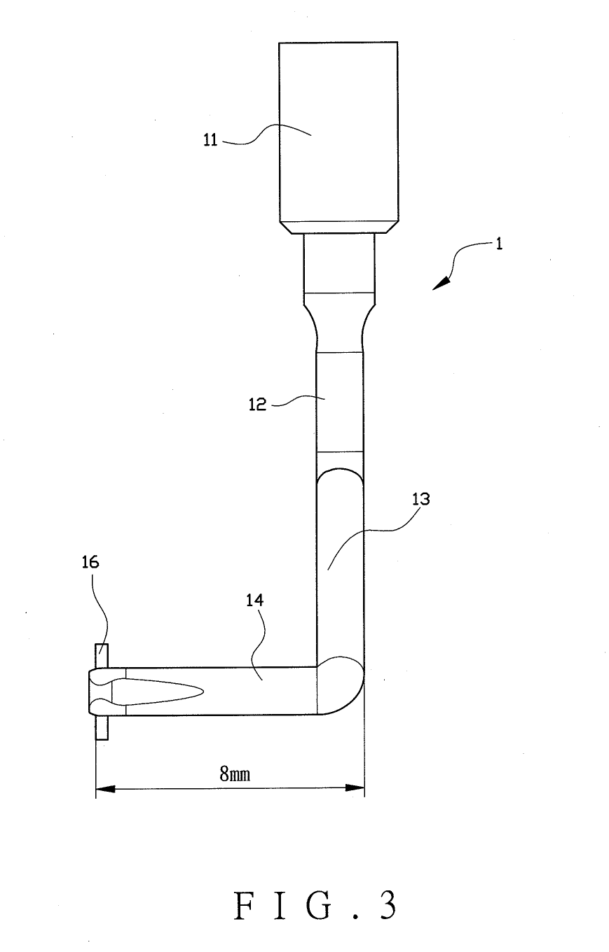

Referring to FIGS. 1 to 3, a preferred embodiment of the ultrasonic osteotome 1 of the present invention comprises a connector 11, a first arm 12, a second arm 13, a third arm 14, a fourth arm 15 and a cutting head 16. A coordinate system comprises an X axis, a Y axis and a Z axis, wherein the X axis, the Y axis and the Z axis are perpendicular to each other and intersect at an original point. The connector 11 extends along the Y axis. The first arm 12 extends along the Y axis and is connected to the second arm 13. The second arm extends along the plane with the X and Y axes. An angle between the first and second arms 12, 13 is within the range between 125 to 145 degrees, preferably is 135 degrees. The third arm 14 extends along the Z axis and is connected to the second arm 13 and the fourth arm 15. Wherein, the second arm extends toward positive direction of the X axis to connect with the first arm. The fourth arm 15 extends along the negative direction of the X axis and is connected to the cutting head 16 which is parallel to the plane with the X and Y axes. The length of the osteotome 1 without the connector 11 in the Y axis is 25.02 mm. The length of the osteotome 1 in the X axis is 10 mm. The length of the osteotome 1 in the Z axis is 8 mm. The length of the first arm 12 in the Y axis is 9.91 mm, and the length of the second arm 13 in the Y axis is 10.03 mm. The length of the cutting head 16 in the X axis is 4 mm.

For further explanations, there are other sizes of the osteotome 1. The length of the osteotome 1 without the connector 11 in the Y axis is 20.016 mm to 30.24 mm. The length of the osteotome 1 in the X axis is 8 mm to 12 mm. The length of the osteotome 1 in the Z axis is 6.4 mm to 9.6 mm. The length of the cutting head 16 in the X axis is 3.2 mm to 4.8 mm.

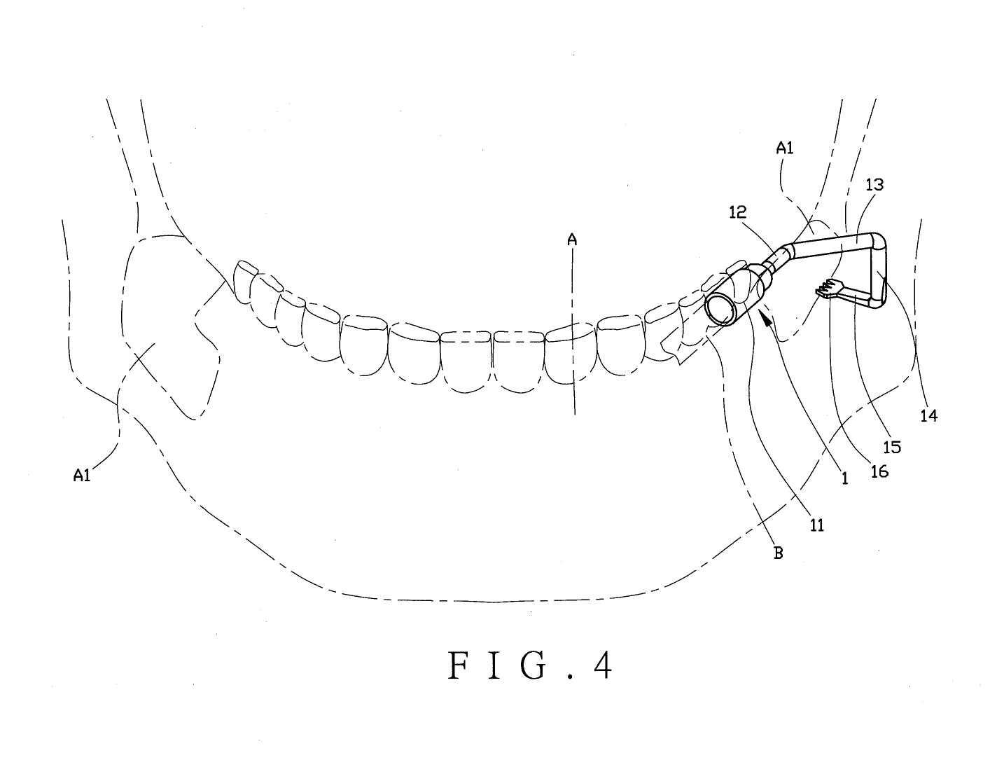

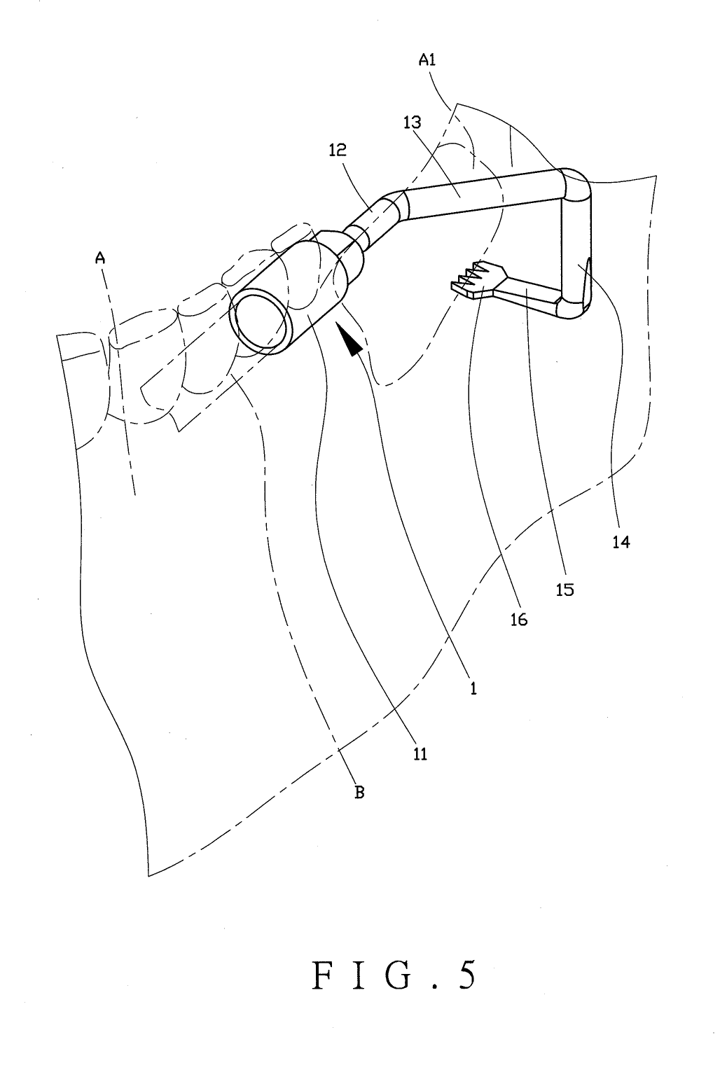

As shown in FIG. 4, when the operation is applied to cut the cutting area A1 of the mandible A, the connector 11 of the osteotome 1 is connected to a ultrasonic osteotome machine B, and the fourth arm 15 connected with the cutting head 16 is inserted into the oral cavity. Because the first, second and third arms 12, 13, 14 are connected in different directions, as shown in FIG. 5, after the osteotome 1 is inserted into the oral cavity, the first, second and third arms 12, 13, 14 are located away from the mandible A so as to prevent from touching the mandible A directly. Further, because of the modified structure and special specifications of the osteotome 1, the osteotome 1 is easily inserted into the oral cavity and directly reaches the cutting area A1 on the left side of the mandible A to proceed the horizontal cutting to the cutting area A1 so as to reach a better cutting effect. Additionally, the osteotome 1 is designed to prevent from hitting the patient's cheeks so as to reduce uncomfortable feeling of the patients.

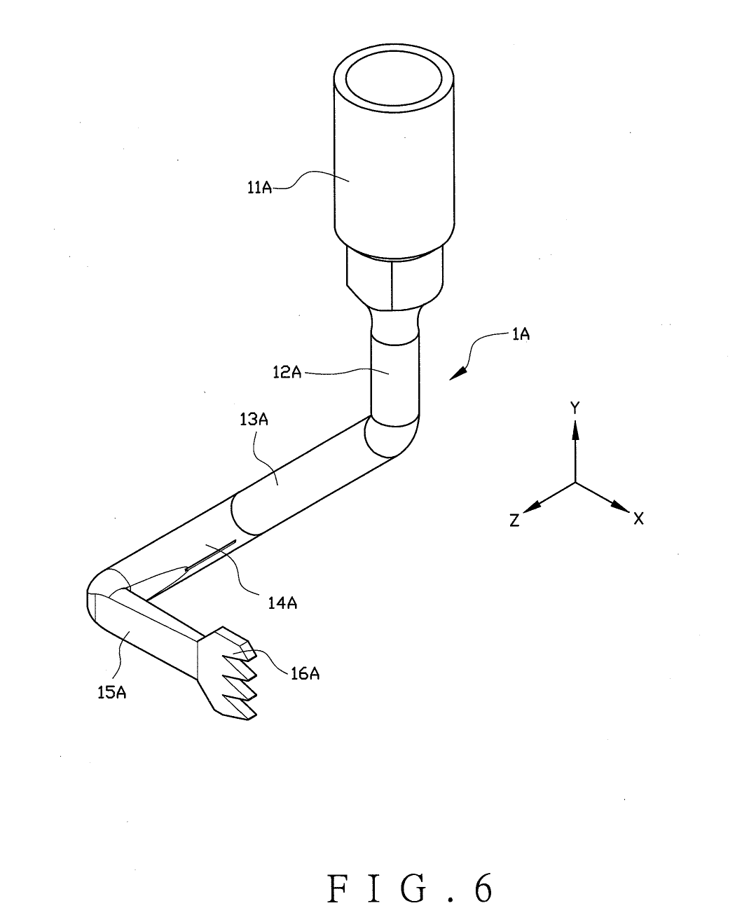

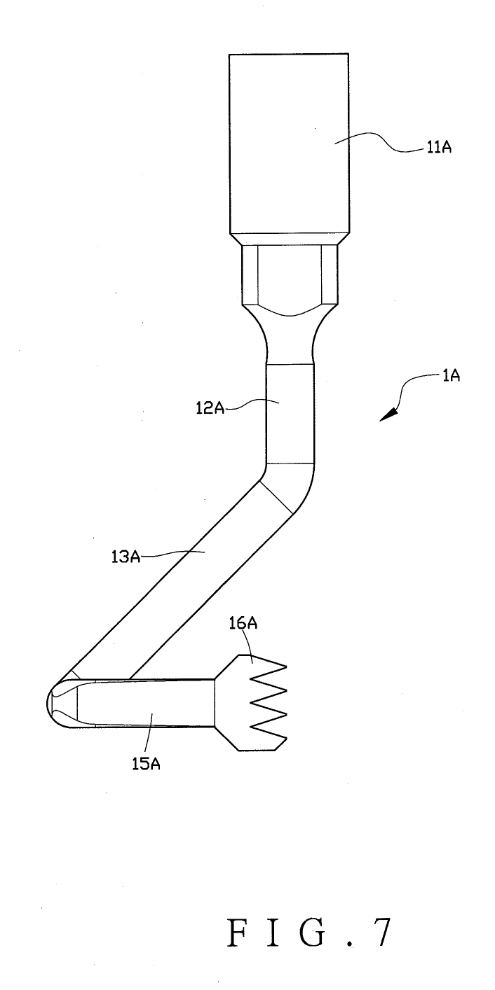

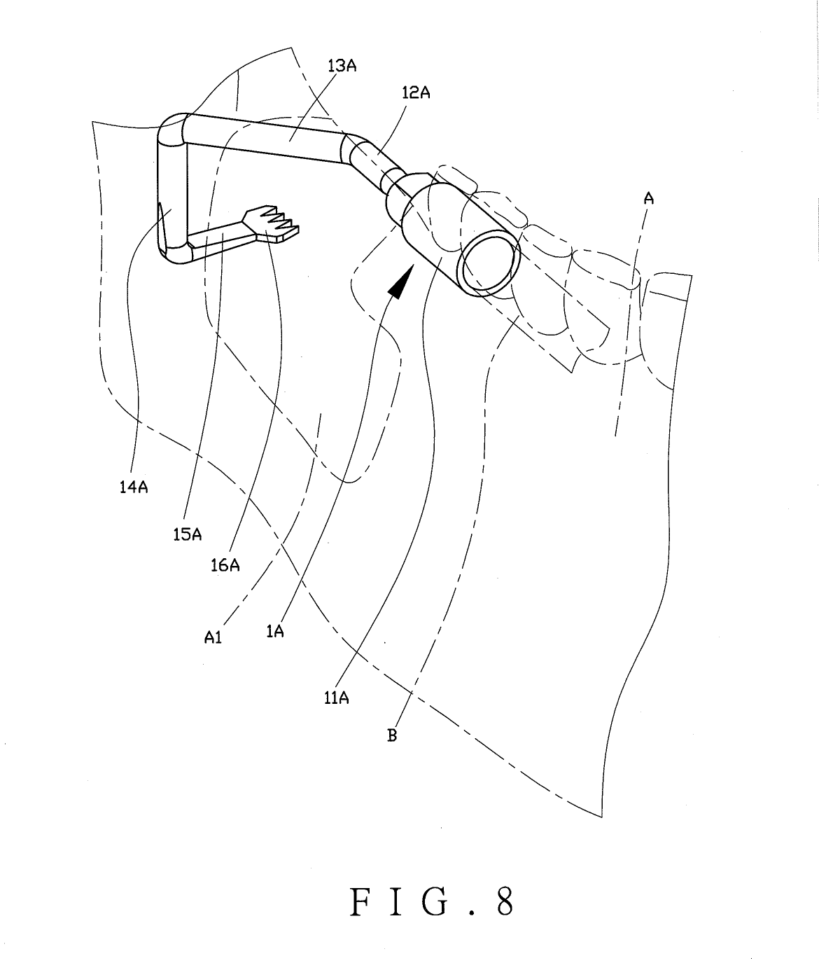

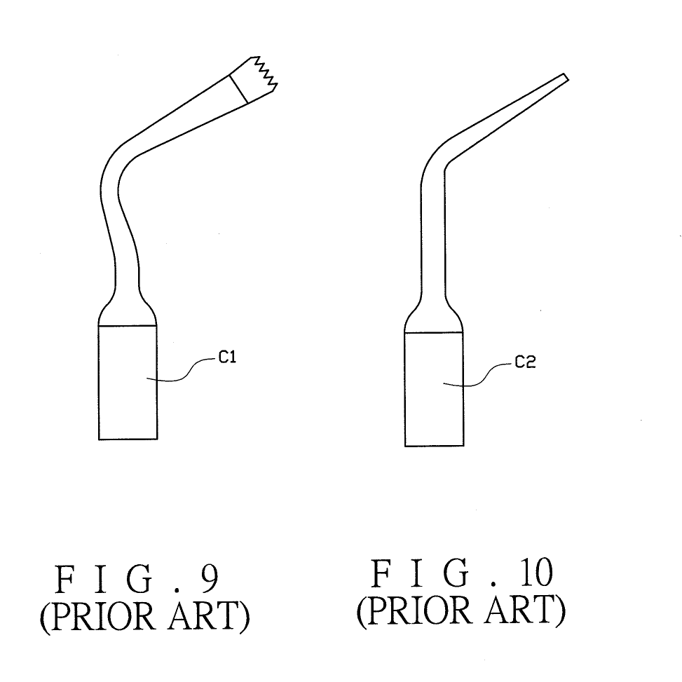

As shown in FIGS. 6 and 7, another preferred embodiment of the present invention shows that the ultrasonic osteotome 1A of the present invention comprises a connector 11A, a first arm 12A, a second arm 13A, a third arm 14A, a fourth arm 15A and a cutting head 16A. Wherein, the second arm extends toward negative direction of the X axis to connect with the first arm. The fourth arm 15A extends along the positive direction of the X axis and is connected to the cutting head 16A. As shown in FIG. 8, when the osteotome 1A is inserted into the oral cavity, the cutting head 16A directly reaches the cutting area A1 on the right side of the mandible A to proceed the horizontal cutting to the cutting area A1.

While we have shown and described the embodiment in accordance with the present invention, it should be clear to those skilled in the art that further embodiments may be made without departing from the scope of the present invention.

What is claimed is:

1. An osteotome comprising:
a connector, a first arm, a second arm, a third arm, a fourth arm and a cutting head, a coordinate system having an X axis, a Y axis and a Z axis, the X axis, the Y axis and the Z axis being perpendicular to each other and intersecting at an original point, the connector extending along the Y axis, the first arm extending along the Y axis and connected to the second arm, the second arm extends along a plane with the X and Y axes, an angle between the first and second arms within a range between 125 to 145 degrees, the third arm extending along the Z axis and connected to the second arm and the fourth arm, the fourth arm extending along the X axis and connected to the cutting head which is parallel a plane with the X and Y axes.

2. The osteotome as claimed in claim 1, wherein the second arm extends toward positive direction of the X axis to connect with the first arm, the fourth arm extends along a negative direction of the X axis and is connected to the cutting head.

3. The osteotome as claimed in claim 1, wherein the second arm extends toward negative direction of the X axis to connect with the first arm, the fourth arm extends along a positive direction of the X axis and is connected to the cutting head.

4. The osteotome as claimed in claim 2, wherein a length of the osteotome without the connector in the Y axis is 20.016 mm to 30.24 mm, a length of the osteotome in the X axis is 8 mm to 12 mm, a length of the osteotome in the Z axis is 6.4 mm to 9.6 mm, a length of the cutting head in the X axis is 3.2 mm to 4.8 mm.

5. The osteotome as claimed in claim 4, wherein the length of the osteotome without the connector in the Y axis is 25.02 mm, the length of the osteotome in the X axis is 8 mm, the length of the osteotome in the Z axis is 10 mm, the length of the cutting head in the X axis is 4 mm.

6. The osteotome as claimed in claim 5, wherein a length of the first arm in the Y axis is 9.91 mm and a length of the second arm in the Y axis is 10.03 mm.

7. The osteotome as claimed in claim 1, wherein the angle between the first and second arms is 135 degrees.

8. The osteotome as claimed in claim 3, wherein a length of the osteotome without the connector in the Y axis is 20.016 mm to 30.24 mm, a length of the osteotome in the X axis is 8 mm to 12 mm, a length of the osteotome in the Z axis is 6.4 mm to 9.6 mm, a length of the cutting head in the X axis is 3.2 mm to 4.8 mm.

9. The osteotome as claimed in claim 8, wherein the length of the osteotome without the connector in the Y axis is 25.02 mm, the length of the osteotome in the X axis is 8 mm, the length of the osteotome in the Z axis is 10 mm, the length of the cutting head in the X axis is 4 mm.

10. The osteotome as claimed in claim 9, wherein a length of the first arm in the Y axis is 9.91 mm and a length of the second arm in the Y axis is 10.03 mm.
